(19) United States
(12) Patent Application Publication
(10) Pub. No.: US 2023/0027710 A1
(43) Pub. Date: Jan. 26, 2023

(54) METHOD FOR PROVIDING SUPPORT TO MAINTAIN AND IMPROVE HEALTH OF CONSUMERS BY USING HEALTH PREDICTION MODEL THROUGH RECOGNITION OF THEIR HEALTH CONDITION, AND METHOD FOR PROVIDING INFORMATION

(71) Applicant: OTSUKA PHARMACEUTICAL CO., LTD., Tokyo (JP)

(72) Inventors: Yasushi NAKAGAWA, Osaka (JP); Naoyuki DEGUCHI, Osaka (JP); Keisuke HAMAMOTO, Osaka (JP); Masamichi TOBA, Osaka (JP); Soh IWASHITA, Osaka (JP); Mina TSUBOUCHI, Osaka (JP); Kyungtak KWAK, Osaka (JP); Miho YOSHIE, Osaka (JP)

(73) Assignee: OTSUKA PHARMACEUTICAL CO., LTD., Tokyo (JP)

(21) Appl. No.: 17/827,253

(22) Filed: May 27, 2022

(30) Foreign Application Priority Data
May 31, 2021 (JP) 2021-091759

(51) Int. Cl. G16H 50/20; G16H 50/70

(52) U.S. Cl. CPC G16H 50/20; G16H 50/70

(57) ABSTRACT

In an information providing method, purchase data indicating a purchase history of a control customer is acquired, and a merchandise category to which at least one merchandise belongs is determined from the purchase data. In this method, an extent of a potential health symptom of a target customer is further predicted using a health predictive model having the merchandise category to which at least one merchandise included in the purchase history belongs and an extent of the health symptom correlated therein, and the merchandise category. In the method, health information indicating the extent of the predicted health symptom is provided to the target customer.

This application claims priority to Japanese Patent Application No. 2021-091759 filed on May 31, 2021, entitled “Method for providing support to maintain and improve health of consumers by using health prediction model through recognition of their health condition, and method for providing information”. The content of the aforementioned patent application is incorporated by reference herein in its entirety.

### BACKGROUND

### 1. Technical Field

The present disclosure relates to a method for building a health support model that acquires a health condition of a person of an ordinary life and that maintains and improves health of the person of an ordinary life using a health predictive model, and an information providing method.

### 2. Related Art

The life environment has recently been drastically changing throughout the world such as the depopulation, the economic depression, occurrence of natural disasters, and prevalence of infectious diseases. On the other hand, opportunities to actively use artificial intelligence (AI) are increasing, and development and prevalence of the fifth generation mobile communication system, and the like are advancing. In this change of the life environment, the infrastructures supporting the general society start to transition to an industrial frame in which the infrastructures actively use technologies and link to each other based on data, and visualization of information on the social activities and behaviors has started.

With the growing interest in health, it is also required to adapt the efforts for health management to the social movement. The “passive healthcare” has been predominant so far with which a person receives medical treatment after being affected with a disease and the start of symptomatic worsening. It is however considered that such medical cares will also be conducted from now on, as “proactive medical care” with which a healthy person acquires data on the person at home and the data is collected to present the health condition of each person as data to thereby conduct look-ahead health management and “preventive medical care” to conduct disease prevention. It can also be assumed that these medical cares will be followed by “predictive medical care” to conduct prediction of future diseases and calling attention to health.

Japanese Laid-Open Patent Publication No. 2017-006745 discloses a technique according to which a personal health record (PHR) including genome information of each user is accumulated in a large-scale genome cohort database and a cohort analysis is conducted for this PHR big data. With the technique of the above '745 publication, the correlation is derived between the combination of the genome type and the lifestyle type, and the health risk (that is, a disease onset risk), based on the result of the analysis, and the future health risk of a user is estimated.

### SUMMARY OF INVENTION

Various methods can be considered for providing information on the health risk. When the information providing method is diversified, a user tend to easily acquire information needed by the user. A new method for providing information on the health risk is required.

One object of the present invention is to provide a technique capable of calling attention for subjects to the predetermined diseases, disorders that are not diseases but that each are associated with a symptom obstructing their daily life, and the like.

The method according to an exemplary embodiment of the present invention is a method for building a health predictive model. In this method, purchase data indicating a purchase history of each of plural controls is acquired and symptom data indicating an extent of a health symptom collected from each of the plural controls is acquired. The symptom data includes the result of a questionnaire on the health condition, the result of a checkup, and the like. Machine learning is performed using the purchase data as an explanatory variable and the symptom data as an objective variable, and a merchandise category to which at least one merchandise included in the purchase history belongs and the extent of the health symptom are correlated with each other to build a health predictive model that urges the subject to improve the health thereof.

Another method according to an exemplary embodiment of the present invention is an information providing method. In this method, purchase data indicating a purchase history of a target customer is acquired and a merchandise category to which at least one merchandise belongs is determined from the purchase data. An extent of a potential health symptom of the target customer is predicted using a health predictive model correlating therein a merchandise category to which at least one merchandise included in the purchase history belongs and an extent of the health symptom with each other, and the merchandise category. Health information indicating the predicted extent of the health symptom and the health support is provided to the target customer.

According to the exemplary embodiment of the present invention, the technique capable of calling attention for subjects to predetermined diseases, disorders that are not diseases but that each are associated with a symptom obstructing their daily life, and the like can be provided.

### BRIEF DESCRIPTION OF DRAWINGS

FIG. 1 is a diagram depicting an overview of a learning system that performs machine learning using an information processing device and that builds a predictive model.

FIG. 2 is a diagram depicting an overview of a prediction system that performs health prediction for a customer using the information processing device having the predictive model built therein.

FIG. 3 is a diagram depicting the learning system that builds a menopause predictive model related to the health of women.

FIG. 4 is a diagram depicting a specific example of training data 12.

FIG. 5 is a diagram depicting an example of a merchandise group selected significantly more by subjects in an H-layer of subjects in the H-layer, an M-layer, and an L-layer.

FIG. 6 is a diagram depicting a relations between the menopause symptoms and the associated health problems, found from items purchased by the subjects in the H-layer.

FIG. 7 is a diagram depicting examples of a merchandise group for women belonging to the H-layer and having a menopausal symptom.

FIG. 8 is a hardware configuration diagram of an information processing device 10 used when machine learning is performed.

FIG. 9 is a flowchart depicting the procedure for a learning process performed by a CPU 21 of the information processing device 10.

FIG. 10 is a hardware configuration diagram of an information processing device 30 that includes a duplicated predictive model 27.

FIG. 11 is a flowchart depicting the procedure for a prediction process for compatibility, that is performed by a CPU 41 of the information processing device 30.

FIG. 12 is a diagram depicting an example of a display of a chat performed between a customer and the information processing device 30 displayed when health information is notified on a smartphone 38.

FIG. 13 is a diagram depicting the details of each of health (a menopause symptom), a sleeping condition, an exercise condition, an immune state, a body water state, and a nutritional state of women.

### DETAILED DESCRIPTION

An exemplary embodiment of the present invention will be described below in detail with reference to the accompanying drawings. Unnecessarily detailed description may however not be made. For example, detailed description for an already well-known item and duplicative description for substantially same configurations may not be made. This is to avoid causing the description below to be unnecessarily redundant and to facilitate the understanding of those skilled in the art. The inventor provides the accompanying drawings and the following description for those skilled in the art to fully understand the present disclosure, and does not intend to limit the subject matter described in the claims by the drawings and the description.

The configuration and the operation of the embodiment described below are exemplary. The present disclosure is not limited to the configuration and the operation of the embodiment described below. The drawings are schematic drawings and are not necessarily strictly depicted. In the drawings, substantially same configurations are denoted by the same reference numerals, and duplicative description will not be made or will be simplified.

### Embodiment

The inventor developed a system to predict the current health condition of a customer and, in the case where the customer is affected by a disease or where it is forecasted that the customer is affected by a disease, the health risk thereof, from the behaviors of the customer, and to provide information on the health risk. The behaviors of each customer can be acquired using a purchase history that shows, for example, what merchandises are purchased at drugstores. From the statistical view point, the purchased merchandises can be correlated with plural persons of ordinary life (including a disease patient), the health condition of the customer, diseases, and the disease risk. It is considered that various types of merchandise can establish such correlations such as foods, beverages, and health appliances. It is considered that, among these, foods and/or beverages more directly reflect the influence of the health condition, and foods and/or beverages are suitable as the merchandises with which the health condition and the like of the customer are correlated. The inventor realized such correlations by collecting data from about 30,000 subjects, conducting machine learning using data of about 3,000 subject thereof, and building a health predictive model (hereinafter, referred to simply as “predictive model”). As a result, prediction is enabled for the health risk of each customer.

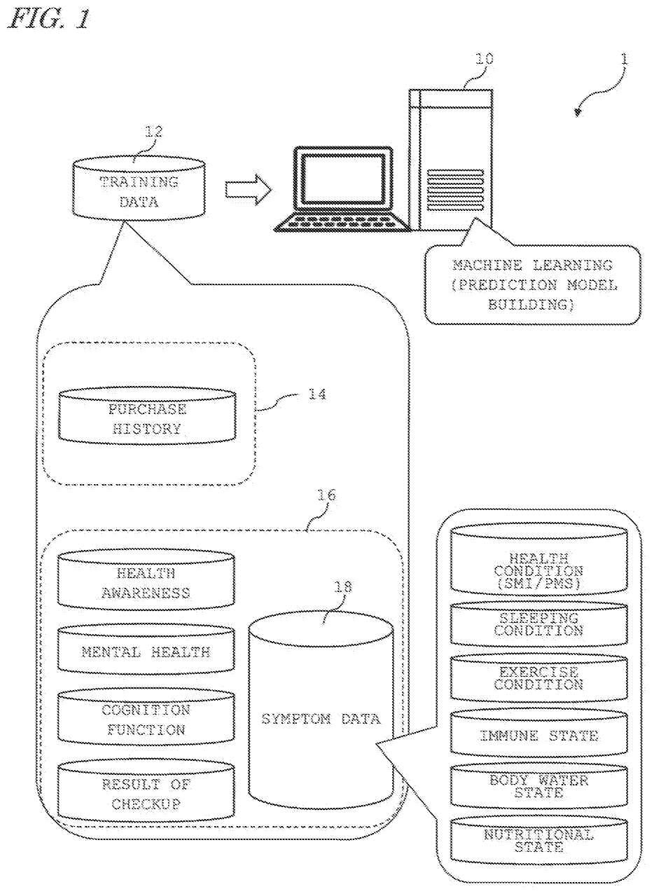

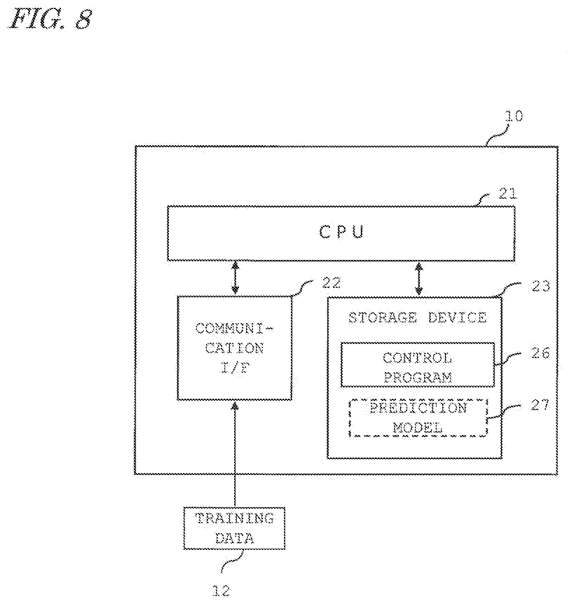

FIG. 1 depicts an overview of a learning system that performs machine learning using an information processing device and that builds a predictive model. The information processing device 10 of the learning system 1 may be, for example, a PC or a server computer. The server computer may be operated on premise by a system provider or may be provided as a cloud service. The specific configuration of the information processing device 10 will be described later with reference to FIG. 8.

The information processing device 10 performs the machine learning using training data 12 that includes various types of data collected from many subjects to thereby build the predictive model. The training data 12 includes one or more pieces of behavioral data 14 on the behavioral algorism of the subjects, and a health related data group 16 that is data on the health of the subjects.

The behavioral data 14 is, for example, purchase data indicating a purchase history of merchandises at drugstores of each of the subjects. The purchase data may include not only the purchase history of the merchandises but also history of receiving services such as, for example, massage, and acupuncture and moxibustion therapy. The purchase data may be identified using the settlement information managed by retailers or may be identified using, for example, the settlement information of credit cards or information on payments to financial institutions. As above, foods and/or beverages are suitable as the merchandises with which the health condition of the customer, and the like are correlated, and it is therefore preferred that the purchase history of foods and/or beverages be included as purchase data.

The health related data group 16 is an aggregate of pieces of data on the health awareness, the mental health, the cognition function, the result of a checkup, and the past medical history and complications, in addition to symptom data 18 on the health of each of the subjects. An example of the symptom data 18 is data indicating the symptom of each of the health condition, the sleeping condition, the exercise condition, the immune state, the body water state, and the nutritional state. The “symptom” may be a subjective symptom or may be a symptom recognized as results of measurement.

The training data 12 is roughly classified into explanatory variables and objective variables. For example, the symptom data 18 may be used as an objective variable and the pieces of data other than the symptom data 18 may be used as an explanatory variable. The behavioral data 14 on the behavioral algorism may be used or at least one type of data selected from the health awareness, the mental health, the cognition function, and the result of a checkup in the health related data group 16 may be used as the explanatory variable.

The inventor employed logistic regression as the method for the machine learning. The logistic regression is however an example. The accuracy may be improved by employing logistic regression using an L1-regularization method, or logistic regression using an L2-regularization method instead of the L1-regularization method (ridge regression) may be employed. An elastic net using both of the L1-regularization method and the L2-regularization method may also be used. A method other than the logistic regression may be used such as, for example, the random forest, the decision tree, the gradient boosting, the support vector regression, the linear regression, the partial least square (PLS) regression, the Gaussian process regression, or the neural network.

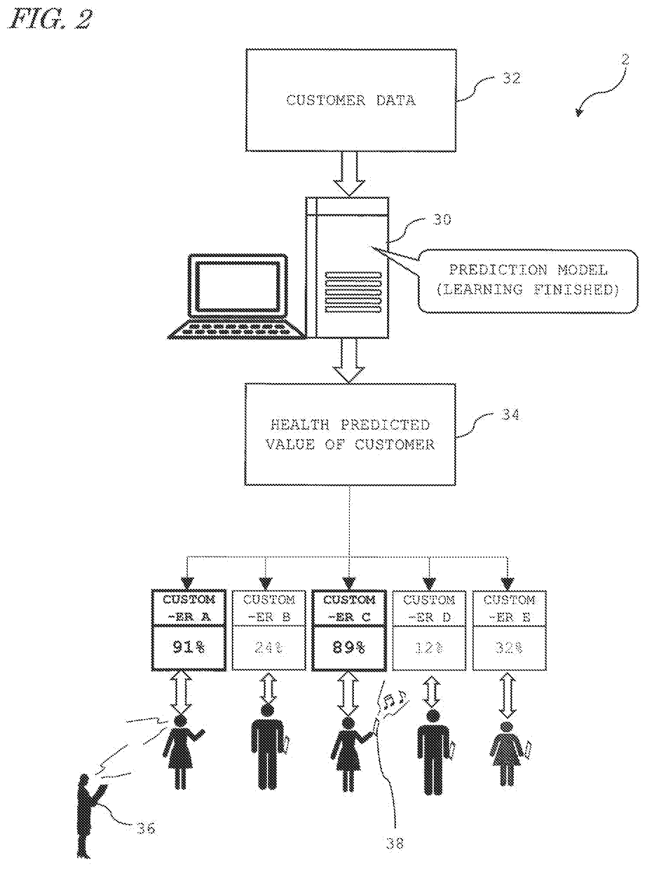

FIG. 2 depicts an overview of the prediction system that performs health prediction for a customer using the information processing device having the predictive model built therein. The predictive model that finishes learning and that is built in the learning system 1 depicted in FIG. 1 is mounted on an information processing device 30 of a prediction system 2. It is assumed that the information processing device 30 is a device different from the information processing device 10 while the information processing devices 30 and 10 may be one same device. In the former case, parameters specifying the predictive model that are extracted from the information processing device 10 are duplicated in the information processing device 30.

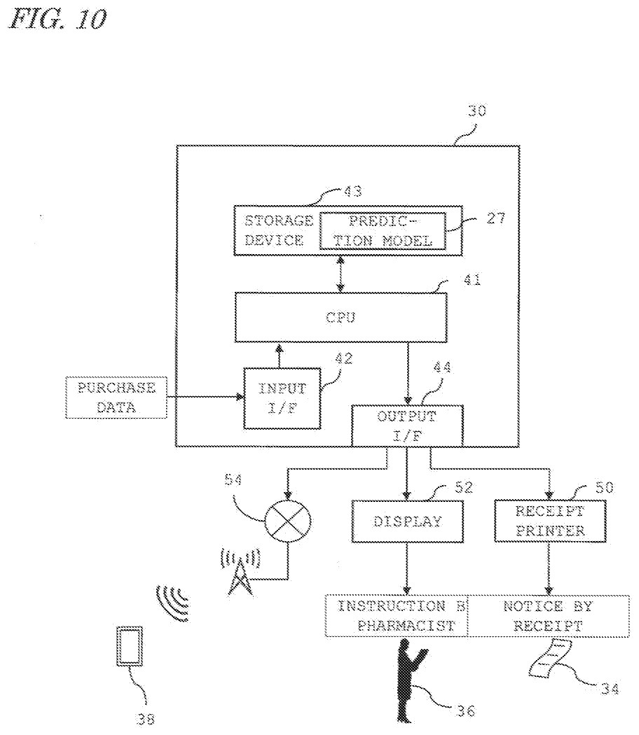

The information processing device 30 may be a commercially available computer system such as, for example, a desktop PC, a notebook PC, or a tablet PC. The computer system may be disposed in a business establishment of the business operator operating the prediction system 2 or may be provided as a cloud service. In the latter case, a PC or the like that can mutually communicate with the cloud service only has to be disposed in the business establishment. Typically, the business operator is a medical doctor and/or a pharmacist, and the business establishment is a hospital, a clinic, a pharmacy, and/or a drugstore. Other business operators and other business establishments can also use the prediction system 2. The detailed configuration of the information processing device 30 will be described later with reference to FIG. 10.

For using the prediction system 2, data 32 of the customer whose health is desired to be predicted (customer data) is prepared. The prepared customer data 32 is the data that corresponds to the explanatory variable of the training data 12 used when the predictive model is built.

The information processing device 30 outputs the symptom data by inputting the customer data 32 of a customer into the predictive model. The symptom data is, for example, a health predictive value 34 of the customer and, more specifically, is a value indicating the possibility and/or the extent that the customer is affected by a specific disease. FIG. 2 presents the possibility indicating that each of customers A to E is affected by a disease (the having-disease rate) in “%”. Depending on the type of the disease, only male persons, only female persons, or both of male and female persons may be the targets. The depicted example presents the extent of being affected by a disease by which both of male and female persons may be affected such as, for example, a cold.

The depicted example presents customers A and C who each presented the having-disease rate equal to a predetermined threshold value or higher. Among these, for the categories each having a high health predictive value, a retailer or a manufacturer 36 can contact the customers and point out the possibility of being affected to propose an employable procedure while the person of an ordinary life is not aware of the disease or an exposed symptom is mild. Like the customer C, a notice is transmitted together with an alarm sound to a smartphone 38 of the customer C. With the notice, the customer C can learn the possibility of being affected and can receive the proposal for the employable procedure while the symptom is mild.

The exemplary embodiment of the present invention will be described below exemplifying health of women as the health condition, more specifically, the menopausal symptom represented by a simplified menopausal index (SMI). Depending on the country or the region, any one or more of other indexes relating to menopause is/are usable as necessary such as, for example, the Menopause Rating Scale, the Kupperman index, Green Climacteric Scale, the premenstrual symptoms screening tool (PSST), Menopause Rating Scale (MRS), the Women's Health Questionnaire (WHQ), Visual Analogue Scale (VAS), Hot Flash Related Daily Interference Scale (HFRDI), Hot Flash Composite Score (HFCS), and Menopause-Specific Quality of Life (MENQOL).

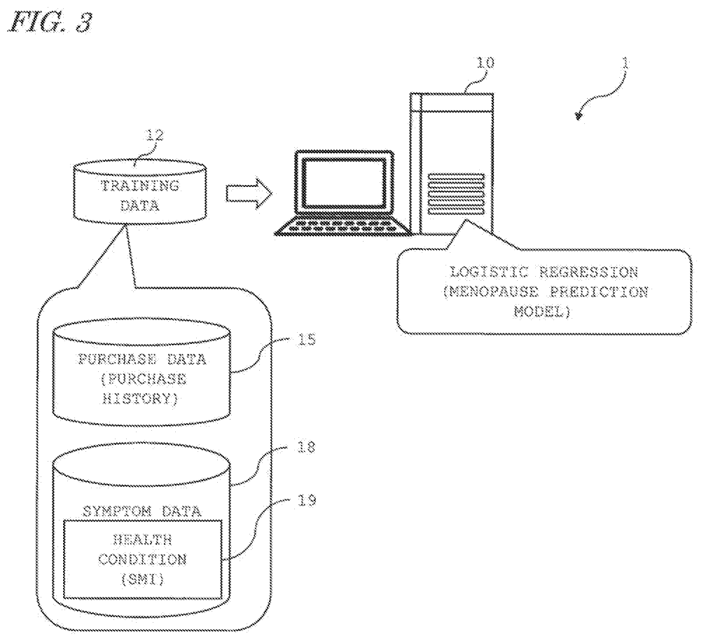

FIG. 3 depicts the learning system that builds a menopause predictive model related to the health of women. In this example, purchase data 15 indicating the purchase history as the explanatory variable, and the simplified menopausal index (SMI) 19 indicating the health condition of women that is one of the pieces of symptom data 18 as the objective variable are included as the training data 12. The purchase data 15 and the simplified menopausal index (SMI) 19 are collected from each of the subjects.

For example, panel data having purchase histories accumulated therein that are provided by a research company is employable as the purchase data 15. The panel data is data acquired by scanning a trade name of a merchandise by a subject who is the purchaser when the merchandise is purchased. When the subject scans the merchandise using a smartphone of the subject or a barcode reader lent to the subject, the merchandise is accumulated being correlated with the attribute (a company employee, a student, a housewife, or the like) of the subject set in advance, information on the place of the purchase, and the like. The type of the purchaser, the purchased item, the quantity of the item, and the store of the purchase are thereby acquired. It is not essential to employ the purchase data 15 as the explanatory variable as above, and various types of data group exemplified in FIG. 1 may be employed.

The simplified menopausal index (SMI) 19 is acquired by causing each of the subjects to perform in advance the concerned items according to the extent of each of the listed various types of symptom, and converting the result thereof into scores. The simplified menopausal index (SMI) 19 is regarded as an index that reflects the symptoms specific to Japanese women in the menopausal period. Therefore, depending on the country or the region, any one or more of other indexes related to the menopause symptoms is/are usable as necessary such as, for example, the Menopause Rating Scale, the Kupperman Index, Green Climacteric Scale, the premenstrual symptoms screening tool (PSST), Menopause Rating Scale (MRS), the Women's Health Questionnaire (WHQ), Visual Analogue Scale (VAS), Hot Flash Related Daily Interference Scale (HFRDI), Hot Flash Composite Score (HFCS), and Menopause-Specific Quality of Life (MENQOL).

The information processing device 10 employs the logistic regression as the method for the machine learning. The logistic regression refers to the method according to which an input is received, the probability that the input corresponds (or the probability that the input does not correspond) to an event is calculated, and whether the input corresponds to the event is thereby determined. A model of the logistic regression is explained as Equation (1) below as an example. “i” represents an i-th target, “p” represents the probability that the event of the objective variable occurs, “b₁, b₂, . . . bₙ” represent partial regression coefficients, “b₀” represents a constant term, and “x₁, x₂, . . . xₙ” represent explanatory variables. The left member is a natural logarithm. Equation 1 is referred to as “logit function”.

$$[ Eq . 1 ]$$

$$log \frac{{p}_{i}}{1 - {p}_{i}} = {b}_{0} + {b}_{1} {x}_{1} + {b}_{2} {x}_{2} + … + {b}_{n} {x}_{n} \qquad ( 1 )$$

Equation (1) can be transformed into Equation (2) that represents the probability p.

$$[ Eq . 2 ]$$

$${p}_{i} = \frac{1}{1 + {e}^{- z}} \\ ( z = {b}_{0} + {b}_{1} {x}_{1} + {b}_{2} {x}_{2} + … + {b}_{n} {x}_{n} ) \qquad ( 2 )$$

Equation (2) is referred to as “sigmoid function” and is an activating function in a logistic regression model. The partial regression coefficients “b₁, b₂, b₂” in the definition of “z” are each referred to as “weight” and “b₀” is a bias term. When z is input into the sigmoid function, a value from zero to one, that is, a probability value is output.

The sigmoid function acquired using Equation (2) is represented by “ϕ (z)” and is classified into an output y that represents any one of the binary according to the output value therefrom. Equation (3) means that ϕ (z) is classified into the class of one when ϕ (z) is 0.5 or greater, and is classified into the class of zero when ϕ (z) is less than 0.5. In other words, Equation (3) means that ϕ (z) is classified into the class of one when z in Equation (2) is zero or greater, and is classified into the class of zero when z in Equation (2) is less than zero.

$$[ Eq . 3 ]$$

$$y = \{ \begin{matrix}1 ( φ ( z ) \geq 0.5 ↔ z \geq 0 ) \\ 0 ( φ ( z ) < 0.5 ↔ z < 0 )\end{matrix} \qquad ( 3 )$$

The “likelihood” that is used in learning in the logistic regression is next introduced. The likelihood means the plausibility of the condition observed from the result. The likelihood function L representing the likelihood is represented by Equation 4 below. “p(.)” represents a probability value.

$$[ Eq . 4 ]$$

$$L ( ω ) = {{\prod }^{n}}_{i = 1} P ( {y}^{( i )} \vert {x}^{( i )} ; ω ) = {{\prod }^{n}}_{i = 1} {( φ ( {z}^{( i )} ) )}^{{y}^{( i )}} {( 1 - φ ( {z}^{( i )} ) )}^{1 - {y}^{( i )}} \qquad ( 4 )$$

The likelihood function L represents the probability that all are accurately determined. Outputting more accurately the probability of the event that is desired to be predicted is enabled by determining the weight that maximizes the likelihood. For example, the likelihood function L is multiplied by (−1) and the sign is thereby inverted. The likelihood function with the inverted sign is an error function in the logistic regression. To determine the weight with which the error function takes its minimal value, that is, the partial regression coefficients b₁, b₂, . . . bₙ, the error function is partially differentiated for each of b₁, b₂, . . . bₙ, to apply the gradient descent method. The learning of the logistic regression is thereby performed.

A log likelihood function using the natural logarithm of Equation (4) may be used as the likelihood function. The optimal weight can be found by minimizing the function whose sign is inverted by multiplying the log likelihood function by (−1).

The arithmetic algorithm related to the above logistic regression is known, and a software application to perform the machine learning using the algorithm can easily be acquired. The description is as above in principle while, when available software is used, even a person who does not especially know in detail the specific analysis method that uses mathematical expressions can realize the machine learning that uses the logistic regression of this embodiment.

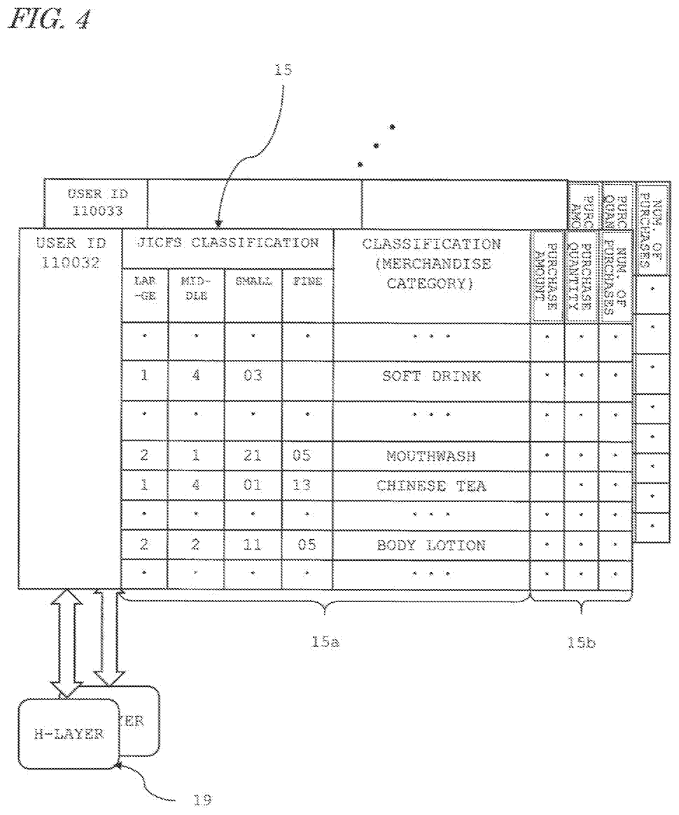

The details of the training data 12 used for the learning of the logistic regression algorithm will be described with reference to FIG. 4.

FIG. 4 depicts a specific example of the training data 12. The training data 12 includes the purchase data 15 and a label 19 that indicates the symptom of the simplified menopausal index SMI to which the subject of the purchase data 15 belongs. In this example, the purchase data 15 indicates the purchase history of the foods and/or the daily commodities purchased by each of the subjects in a specific time period such as, for example, one year. Into the purchase data 15, each item 15a of about 500 items (examples: a soft drink and a mouthwash) of the fine categories included in the large categories of “food” and “daily commodity” in the JICFS classification, and actual result data 15b that is numerical data of the purchase amount, the purchase quantity, the number of purchase sessions, and the like in the time period are input being correlated with each other for each of the IDs of the subjects.

The label 19 indicating the symptom of the simplified menopausal index SMI to which the subject belongs is correlated with each piece of the purchase data 15 in FIG. 4. In this embodiment, the simplified menopausal index SMI has two classes. For example, the two classes are “H” that represents that the menopause symptom is relatively serious and “other-than H”. The “Other-than H” can be finely classified into “M” representing that the menopause symptom is relatively intermediate and “L” representing that the menopause symptom is relatively mild. Hereinafter, the subject layer corresponding to “H” will be referred to as “H-layer”. It can be considered that the customers considered to be highly likely to potentially correspond to the H-layer can be found when a merchandise group often purchased by the customers in the H-layer is prepared as the training data 12.

The purchase amount, the purchase quantity, the number of purchase sessions, and the like of each of the items of the purchase data 15 correspond to an explanatory variable X in Equation (1) above and the like. The likelihood function can be acquired by classifying such that Equation (3) outputs “one” for the H-layer and outputs “zero” for the other-than H-layer. The elements constituting the explanatory variable X are not limited to the purchase data 15. For example, data indicating an age layer may be included. In the case where a disease state different from the menopause symptom is determined, yet another piece of data such as, for example, gender data may be used as the explanatory variable.

To determine the merchandise group often purchased by the customers in the H-layer, the inventor built the predictive model by performing the machine learning using the data of about 3,000 persons as above. As a result, the inventor enabled prediction of the merchandise or the merchandise group purchased by many of the subjects in the H-layer.

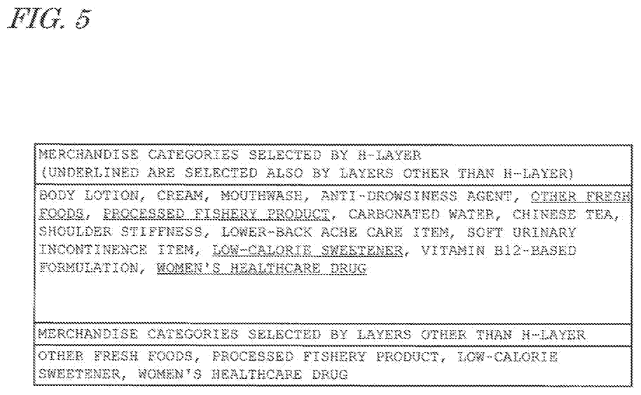

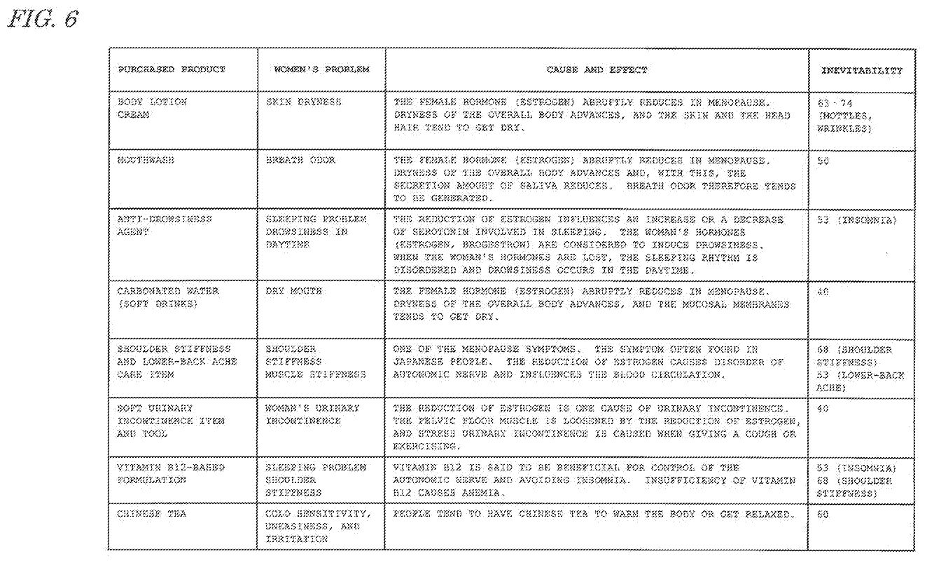

FIG. 5 depicts the merchandise groups selected by many of the user in the H-layer and the layers other than the H-layer. “◯” represents the merchandise group selected by the customers in the H-layer and the layers other than the H-layer. Thick frames and thick letters of these merchandise groups represent the merchandise groups that were selected by many of the customers in the H-layer but not selected so often by the customers in the layers other than the H-layer. FIG. 6 depicts the relation between the menopause symptom and the associated health problems, found from items purchased by the subjects in the H-layer. The values in the rightmost columns in FIG. 6 each show the degree indicating the easiness of being selected by women with the menopause symptom (the deviation). The values are from “Deviations by Item and Symptom Desired to Be Coped with by Even Spending Money—Research Project Report on Understanding of Health, Beauty Care, and Lifestyle of Women—Result of Behavior Observation Research”

It turned out from these results that the subjects in the H-layer actively selected specific merchandises to cope with the menopause symptom. The inventor performed an analysis to find a feature that the subjects purchased the merchandises that coped with skin dryness, internal dryness, and the indefinite complaint.

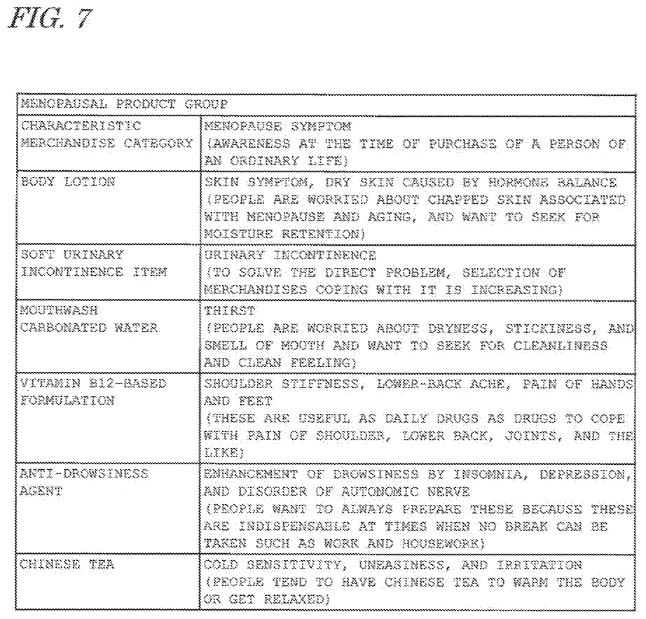

FIG. 7 depicts examples of the merchandise group for women belonging to the H-layer and having the menopausal symptom. When the predictive model is built using the pieces of data classified as above, the merchandises frequently purchased by the subjects in the H-layer compared to the subjects in the layers other than the H-layer can be determined. It can be presumed that the purchasers of those merchandises are the subjects belonging to the H-layer. The inventor built the predictive model using the merchandises whose purchasers were able to be presumed as the subjects belonging to the H-layer, and thereby enabled highly precise presumption of the customers belonging to the H-layer.

The merchandise category to which one or more merchandise(s) belong(s), and the value acquired by calculation according to the amount of money for which the women corresponding to each of the classes purchase the merchandises included in the merchandise category, the quantity of the merchandises, and the number of purchase sessions thereof may be correlated with each other. This correlation may be described as, for example, a function. The predictive model may be acquired as a function. Instead of the classes of H, M, and L layers, a value indicating the possibility of corresponding to the menopause symptom may be employed. The number of classes may be three and may be four or more by more fine division as necessary.

The specific configuration and the specific operation of each of the information processing device 10 (FIG. 1) and the information processing device 30 (FIG. 2) will be described with reference to FIG. 8 to FIG. 11.

FIG. 8 is a hardware configuration diagram of the information processing device 10 used when the machine learning is performed. The information processing device 10 includes a CPU 21, a communication interface (I/F) 22, and a storage device 23.

The CPU 21 is an example of an arithmetic circuit of the information processing device in this embodiment. The CPU 21 realizes predetermined functions including the learning and the implementation of a predictive model 27 by executing a control program 26 stored in the storage device 23. The information processing device 10 realizes the function as the information processing device of this embodiment by execution of the control program 26 by the CPU 21. The control program 26 is an example of the computer program of this embodiment. The arithmetic circuit constituted as the CPU 21 in this embodiment may be realized by each of various types of processor such as an MPU or a GPU, and may be constituted by one or plural processor(s).

The communication interface 22 is, for example, an Ethernet (a registered trademark) communication terminal or a USB (a registered trademark) terminal. Otherwise, the communication interface 22 is a communication circuit that performs communication in conformity to the standard such as IEEE 802.11, 4G, or 5G. The communication interface 22 is connectable to a communication network such as an intranet or the Internet, and receives the training data 12 prepared by the operator of the learning system 1. The information processing device 10 may perform direct communication with another device through the communication interface 22, and may perform communication through an access point.

The storage device 23 is a storage medium that stores therein the computer program and the data necessary for operating the information processing device 10. The storage device 23 may be a solid state drive (SSD) that is, for example, a hard disk drive (HDD) or a semiconductor storage device.

The storage device 23 may include a temporary storage element that includes a RAM such as, for example, a DRAM or an SRAM, and may also function as a working area of the CPU 21. The storage device 23 stores therein the control program 26 to be executed by the CPU 21 and, after the predictive model 27 is built, stores therein a table and/or a parameter group that define the predictive model 27.

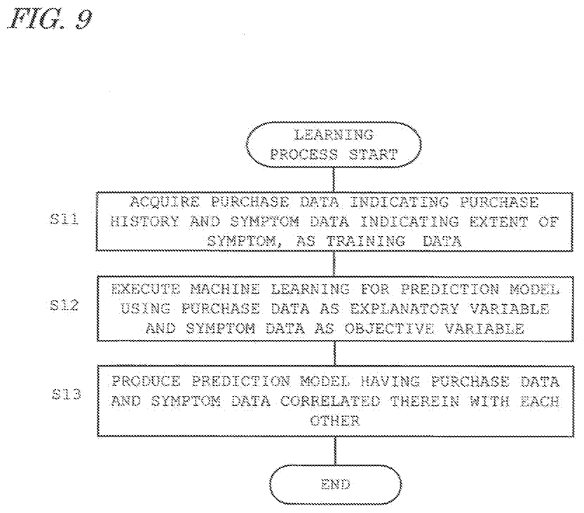

FIG. 9 is a flowchart depicting the procedure for a learning process executed by the CPU 21 of the information processing device 10.

At step S11, the CPU 21 acquires the purchase data 15 indicating the purchase history and the symptom data 18 indicating the extent of the symptom, as the training data 12, through the communication I/F 22. The purchase data 15 includes, for example, the JICFS classification (the classification code) and the merchandise categories. At step S12, the CPU 21 performs the machine learning of the predictive model 27 using the above equations with the purchase data as the explanatory variable and the symptom data as the objective variable.

At step S13, the CPU 21 produces the predictive model 27 that has the purchase data and the extent of the symptom correlated with each other therein.

The predictive model 27 built as above can be used as it is for predicting the compatibility between optional two persons using the information processing device 10. Otherwise, the prediction of the compatibility can be performed also by an optional information processing device other than the information processing device 10 that produces the predictive model 27 by duplicating the data constituting the predictive model 27 in the optional information processing device.

FIG. 10 is a hardware configuration diagram of the information processing device 30 that includes a duplicated predictive model 27. The information processing device 30 calculates a result of the prediction related to the menopause symptom of a customer using the predictive model 27. The information processing device 30 may also be, for example, a PC or a server computer. The information processing device 30 includes a CPU 41, an input interface (I/F) 42, a storage device 43, and an output interface (I/F) 44. The input I/F 42 receives the purchase data of a customer that is desired to be predicted.

The CPU 41, the input I/F 42, and the storage device 43 respectively have the same configurations and functions as those of the CPU 21, the communication I/F 22, and the storage device 43 depicted in FIG. 8. The above description for the CPU 21, the communication I/F 22, and the storage device 23 is therefore substituted for the description for the CPU 41, the input I/F 42, and the storage device 43.

The storage device 23 stores therein a table and/or a parameter group that define the predictive model 27 built by the information processing device 10. The predictive model 27 may be, for example, incorporated as a portion of the computer program executed by the CPU 41, or may be provided as data separated from the computer program.

The output I/F 44 is a communication circuit and/or a communication terminal that are/is connected to various types of output device disposed in the exterior of the information processing device 30. For example, the output I/F 44 is a USB terminal transmitting printing data that indicates the content to be printed by a receipt printer 50. The CPU 41 of the information processing device 30 calculates the prediction result related to the menopause symptom of a customer using the predictive model 27 and transmits the result to the receipt printer 50 to cause the receipt printer 50 to print a receipt 34.

Otherwise, the output I/F 44 may be a video image output terminal connected to a display 52. The CPU 41 of the information processing device 30 calculates the prediction result related to the menopause symptom of a customer using the predictive model 27 and transmits the result to the display 52 to cause the display 52 to display thereon the prediction result. A pharmacist 36 checks the content displayed on the display 52 and proposes, for example, one or plural merchandise(s) purchased by many of the subjects each having the menopause symptom, to a customer having the menopause symptom.

Otherwise, the output I/F 44 may be a communication terminal or a communication circuit that is connected to a communication network 54 and the like and that is capable of performing data communication. In the case where the output I/F 44 is the communication terminal or the communication circuit, the hardware of the output I/F 44 and that of the input I/F 42 may be same as each other and, in this case, the above description for the communication I/F 22 is substituted for the description for the output I/F 44. The output I/F 44 transmits the prediction result to the smartphone 38 of the customer through, for example, a mobile phone line.

A table correlating the prediction result showing the extent of the menopause symptom such as, for example, the H-layer, the M-layer, and the L-layer, and 90%, 50%, and 20%, with the merchandises and the services to be proposed according to each extent may be prepared in advance, and the information processing device 30 may propose the merchandises and the services corresponding to the result together with the prediction result. This table is stored in advance in, for example, the storage device 43.

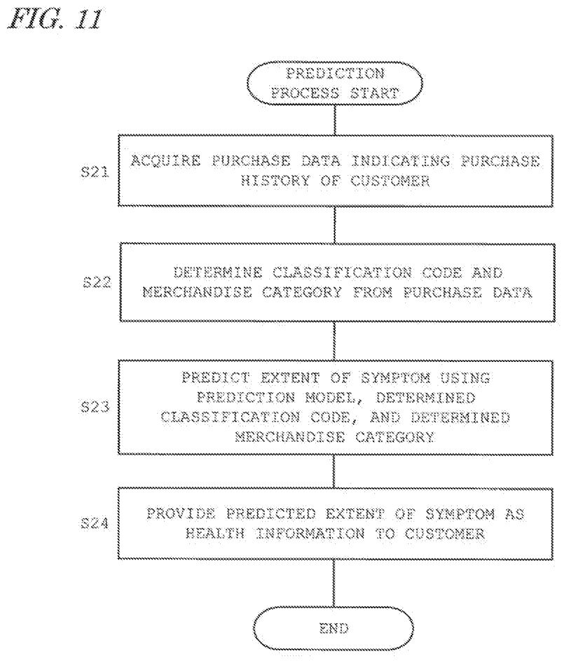

FIG. 11 is a flowchart depicting the procedure for a prediction process performed by the CPU 41 of the information processing device 30.

At step S21, the CPU 41 acquires the purchase data that indicates the purchase history of the customer for whom prediction related to the menopause symptom is made. The acquisition of the purchase data can easily be realized. It is already general that the membership card of a customer is read by a point of sale (POS) system when the customer purchases a merchandise at, for example, a drugstore and a database that links the customer and the purchase history of the customer with each other is thereby built. With the consent of the customer, the purchase data can be input into the information processing device 30 by extracting the purchase data from the database. The purchase data 15 includes, for example, the JICFS classification and the merchandise categories.

At step S22, the CPU 41 determines the JICFS classification (the classification code) and the merchandise category from the purchase data.

At step S23, the CPU 41 predicts the extent of the symptom using the predictive model 27, and the classification code and the merchandise category that are determined.

At step S24, the CPU 41 sends the predicted extent of the symptom as health information to the output I/F 44 to provide the health information to the customer in the form of printing on the receipt 34, a display on the display 52, or a notice to the smartphone 38.

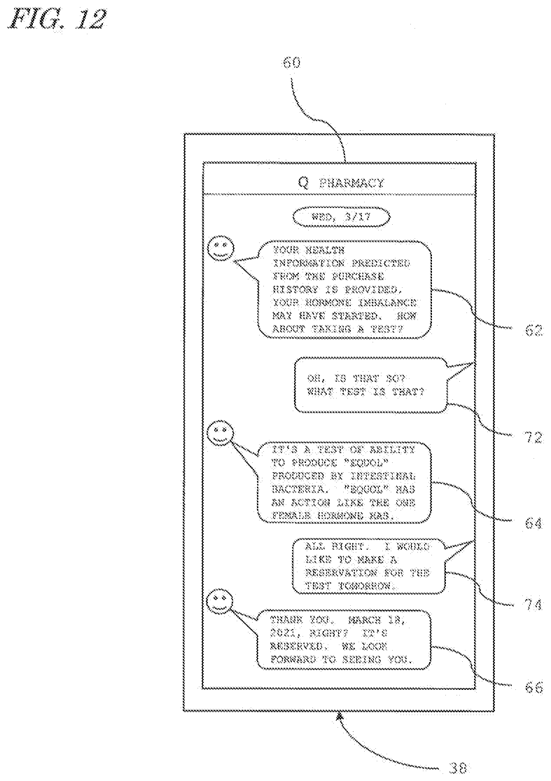

FIG. 12 depicts an example of a display of a chat between the customer and the information processing device 30 displayed in the case where the health information is notified of to the smartphone 38. The technique for a computer to understand the content of a chat to reply has already been realized, and it is therefore assumed in this embodiment that this technique is employed. The specific content of the chat is only an example and the example depicted in FIG. 12 is only one example where a prediction result is transmitted to a smartphone of a customer. It is assumed that the prediction result is sent from a Q pharmacy 60 to a customer.

In a chat 62, the CPU 41 of the information processing device 30 notifies the customer of the fact that the customer has the menopause symptom by informing the customer of the fact, for example, that hormonal imbalance starts, from the prediction result.

In a chat 72, a question is returned by a chat from the customer.

In a chat 64, the CPU 41 of the information processing device 30 recognizes the content of the question and returns an answer to the question to the customer.

When, in a chat 74, the customer hopes for opportunities of a medical examination and a questionnaire based on the prediction result, in a chat 66, the CPU 41 of the information processing device 30 makes reservations for the medical examination and the questionnaire for the customer.

The above process enables the notification of the prediction result related to the menopause symptom to the customer and also the proposal for purchase of merchandises and provision of services.

The predictive model to predict the menopause symptom of women has been described in detail. The inventor however considered that the idea of the present invention was applicable in relation to various types of disease in addition to the menopause symptom of women. For example, in addition to the predictive model of the health of women (the menopause symptom), for the inventor, one or more selected from predictive models of the premenstrual syndrome (PMS, PMDD) is/are usable. The inventor found that such items were each able to independently be predicted as the sleeping condition, the exercise condition, the immune state, the body water state, the nutritional state, a dementia, use of oxygen, the health of blood vessels, the oral hygiene, the health of skin, the health of head hair, the intestinal environment, the mental health, promotion of efficacy of exercise, appetite improvement, the body temperature constancy, and the health of eyes.

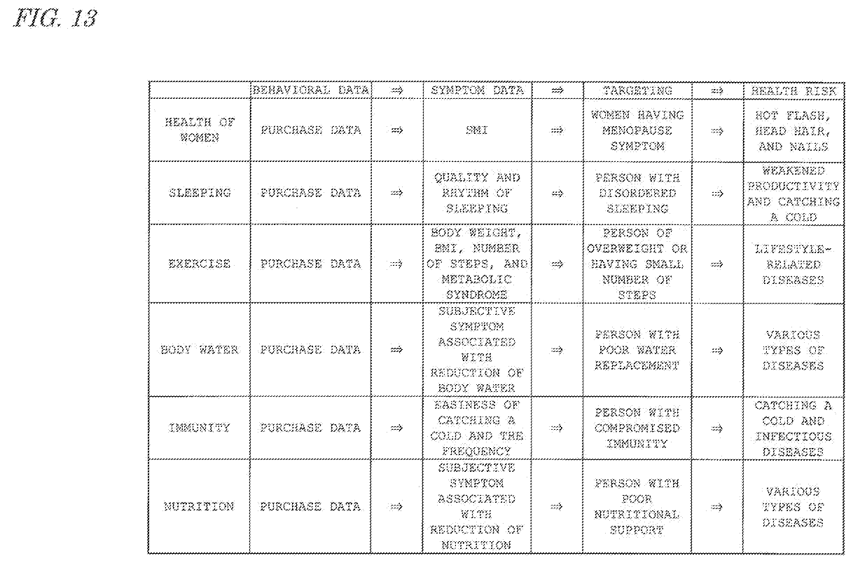

FIG. 13 depicts the details and the like of the health of women (the menopause symptom). The predictive model related to the health risk can be built as a more highly precise predictive model of the health risk related to, for example, the menopause symptom when the machine learning is performed using biological information or the behavioral data as the explanatory variable and the symptom data or disease information as the objective variable, which is made by the applicant. A predictive model can be built for each of, for example, the sleeping condition, the exercise condition, the immune state, the body water state, the nutritional state, a dementia, use of oxygen, the health of blood vessels, the oral hygiene, the health of skin, the health of head hair, the intestinal environment, the mental health, promotion of efficacy of exercise, appetite improvement, the body temperature constancy, and the health of eyes, as the health subject matter. As a result, solutions can be presented such as a proposal of a product A to a customer whose sleep tends to be insufficient and a proposal of a product F to a customer whose nutrition tends to be insufficient. FIG. 13 exemplifies the sleeping condition, the exercise condition, the immune state, the nutritional state, and the body water state, in addition to the health of women.

A building method will be described below for a predictive model related to each of the sleeping condition, the exercise condition, the immune state, the nutritional state, and the body water state.

<Sleeping>

A sleeping condition predictive model related to the sleeping condition is used to predict the extent of the symptom of a person whose sleeping is disordered. To build this sleeping condition predictive model, the behavioral data to be the explanatory variable and the symptom data to be the objective variable are respectively the sleeping condition and/or the sleeping rhythm, and the sleeping time period of each control for each of plural controls.

These variables can be acquired by the method same as that of the example of the menopause symptom. On the other hand, the sleeping condition and/or the sleeping rhythm, and the sleeping time period may be the subjective symptom of each of the controls acquired using a questionnaire or the like, or may each be a measured value recognized as the result of measurement for each of the controls. The sleeping condition is analyzed for each of the items of and the combination of Athens insomnia scale (AIS), Pittsburgh Sleep Quality Index (PSQI), 3-Dimensional Sleep Scale (3DSS), Insomnia Severity Index (ISI), and the extent of the symptom is classified by allocating the health condition of the person of an ordinary life into a category. The sleeping rhythm can be acquired by acquiring the sleeping median time period of each of weekdays and holidays using Munich ChronoType Questionnaire (pMCTQ) and, from the difference therebetween, calculating the social jet lag (SJL).

The sleeping condition predictive model can be built by performing the machine learning using the above. As the method for the machine learning, for example, the logistic regression can similarly be used as in the example of the menopause symptom. The customer having the health risk can be identified and the products each capable of improving the potential health symptom of the target customer whose sleeping condition becomes worse, whose sleeping rhythm is disordered, or whose sleeping time period is improper can be provided, by using the predictive model. The potential health symptom may be one or more selected from worsening of the sleeping condition, the sleeping rhythm, and the improper sleeping time period. The machine learning may be performed including information on the quality of sleeping of each of plural controls.

As to the analysis result, the state where a control currently undergoes a worsened health condition or where a control may undergo a worsened health condition from now on can bibliographically be found from the epidemiological information. It was found that, for example, a person of an ordinary life whose difference in the sleeping rhythm was one hour or longer between weekdays and holidays indicated by the social jet lag had a higher risk of developing dysphoria, the metabolic syndrome, and overweighting than that of a person of an ordinary life whose difference therein was within one hour, and this person can therefore be evaluated to have a high health risk.

<Exercise>

An exercise condition predictive model related to the exercise condition is used to predict the state and the disease risk related to the lifestyle-related diseases. To build the exercise condition predictive model, examples of the behavioral data to be the explanatory variable include whether each of plural controls corresponds to an overweight status, a variation of the body weight in a specific time period such as, for example, a time period from a time in twenties to the present, and/or the extent of the symptom of a person whose exercise amount such as the number of steps is relatively small. The symptom data to be the objective variable is any one or more of the body weight of each of the controls, Body Mass Index (BMI) thereof, the number of steps thereof, and/or the metabolic syndrome diagnostic reference values. The metabolic syndrome diagnostic reference values are one or more of the waist measurement diameter, the threshold values specified for the diagnosis of hyperglyceridemia and/or low-HDL cholesterolemia, the systolic blood pressure and/or the diastolic blood pressure, and the threshold value specified for the diagnosis of fasting hyperglycemia.

The purchase data can be acquired by the same method as that of the example of the menopause symptom. For example, the purchase history of the foods and/or the daily commodities purchased by each of the plural controls in a specific time period such as, for example, one year can be acquired. On the other hand, the symptom data may be the measured values recognized as the result of measuring each of the controls. The extent of the health symptom includes one or more extent(s) selected from insufficient exercise, overweight, and a variation of the body weight in a specific time period.

The exercise condition predictive model can be built by performing the machine learning using the above. As the method for the machine learning, for example, the logistic regression can similarly be used as in the example of the menopause symptom. To the target customers each having a disorder in any one of the body weight, BMI, the number of steps, and/or the metabolic syndrome diagnosis reference values, the products each capable of improving the above can be provided by using the predictive model. The potential health symptom may be one or more selected from insufficient exercise, overweight, and a variation of body weight in a specific time period. The machine learning may be performed including one or more pieces of information of being a smoker or a non-smoker and sedentary behaviors for each of the plural controls.

As to the analysis result, the state where a control currently undergoes a worsened health condition or where a control may undergo a worsened health condition from now on can bibliographically be found from the epidemiology. It has been found that, for example, a person of an ordinary life whose number of steps is small has a higher risk of having the metabolic syndrome than that of a person of an ordinary life whose number of steps is great, and that a person of an ordinary life who is overweight has a higher risk of diabetes than that of a person of an ordinary life who is not overweight, and these persons can each be evaluated to have a high health risk.

<Immunity>

An immune state predictive model related to the immune state is used to predict the extent of a symptom of a person whose body is immunologically compromised. To build the immune state predictive model, the behavioral data to be the explanatory variable and the symptom data to be the objective variable are respectively the purchase data indicating the purchase history for each of the plural controls and the state of the immunological capacity of each of the controls.

The purchase data can be acquired by the same method as that of the example of the menopause symptom. For example, the purchase history of the foods and/or the daily commodities purchased by each of the plural controls in a specific time period such as, for example, one year can be acquired. On the other hand, easiness of catching a cold and/or the frequency thereof may be the subjective symptom of each of the controls acquired by a questionnaire or the like, or may be acquired from the medical consultation history of a medical institution. The extent of the symptom is totaled each in categories of three or more levels ,and the extent of the symptom may be easiness of catching a cold, the SIgA concentration, an allergic symptom, the state of the oral environment, a serious sleeping symptom, an intermediate sleeping symptom, and a mild sleeping symptom.

The immune state predictive model can be built by performing the machine learning using the above. As the method for the machine learning, for example, the logistic regression can similarly be used as in the example of the menopause symptom. The products each capable of improving the potential health symptom of the target customer whose immunological capacity is worsened can be provided by using the predictive model. The potential health symptom may be one or more selected from easiness of catching a cold, the SIgA concentration, an allergic symptom, and the state of the oral environment. The machine learning may be performed including one or more pieces of information selected from the easiness of catching a cold, the SIgA concentration, an allergic symptom, and the state of the oral environment of each of the plural controls.

<Body Water>

A water replacement state predictive model related to the water replacement state is used to assume in advance and to avoid the persons who are unable to normally replace their body water, the health risk caused by insufficient body water, the risk thereof. To build the predictive model, the pieces of data to be the explanatory variables are the serum Na value, the BUN/creatinine ratio, and/or the dehydration evaluation scale, and the subjective symptom associated with insufficient body water of each of the plural controls.

These pieces of information can similarly be acquired as in the example of the menopause symptom. For example, the variation in the body experiences by each of the plural controls in a specific time period such as, for example, one year is acquired using clinical laboratory data acquired in medical checkups and complete medical checkups and separately conducted health questionnaires. On the other hand, the subjective symptom associated with reduction of body water can be acquired using questionnaires and the like. The extent of the symptom can be acquired from the serum Na value, the BUN/creatinine ratio, the water intake amount, the body activity amount, and the color of urine.

The water replacement state predictive model can be built by performing the machine learning using the above. As the method for the machine learning, for example, the logistic regression can similarly be used as in the example of the menopause symptom. The products each capable of improving the potential health symptom of the target customers each having a subjective symptom associated with the reduction of body water can be provided by using the above predictive model. The potential health symptom may be one or more selected from a high value of the serum Na value and a high value of the BUN/creatinine ratio. The machine learning may be performed including one or more pieces of information of the water intake amount, the alcohol intake amount, the body activity amount, and the color of urine of the plural controls.

As to the analysis result, the state where a control currently undergoes a worsened health condition or where a control may undergo a worsened health condition from now on can bibliographically be found from the epidemiological information. It has been found that, for example, a person of an ordinary life whose serum Na value exceeds 142 mEq/L has a higher risk of developing cognitive impairment and a high blood pressure than that of a person of an ordinary life whose serum Na value is lower than 142 mEq/L, and this person can be evaluated to have a high health risk.

<Nutrition>

A nutritional state predictive model related to the nutritional state is used to predict the extent of the symptom of a person who cannot conduct any nutritional support therefor. To build the above nutritional state predictive model, the behavioral data to be the explanatory variable and the symptom data to be the objective data are respectively the purchase data indicating the purchase history of each of the plural controls and the subjective symptom capable of reflecting the nutritional worsening of each of the controls.

The purchase data can be acquired by the same method as that of the example of the menopause symptom. For example, the purchase history of the foods and/or the daily commodities purchased by each of the plural controls in a specific time period such as, for example, one year can be acquired. On the other hand, the subjective symptom capable of reflecting worsened nutrition may be the subjective symptom of each of the controls acquired by a questionnaire and the like, or may be a measured value recognized as the result of measuring each of the controls. The extent of the symptom may be presence or absence of the subjective symptom associated with worsened nutrition, the dietary variety score (DVS), the simplified nutritional appetite questionnaire (SNAQ), a serious sleeping symptom, an intermediate sleeping symptom, and a mild sleeping symptom.

The nutritional state predictive model can be built by performing the machine learning using the above. As the method for the machine learning, for example, the logistic regression can similarly be used as in the example of the menopause symptom. The products each capable of improving the potential nutritional symptom of the target whose nutrition is disordered can be provided. The potential health symptom may be one or more selected from the insufficient nutrition, disproportionate nutrition, and the dietary habit. The machine learning may be performed including one or more pieces of information from insufficient nutrition, disproportionate nutrition, and the dietary habit of the plural controls.

The flowchart executed by the CPU 41 of the information processing device 30 and a flowchart executed by a CPU 121 of an information processing device 100 are realizable each as a computer program.

The exemplary embodiment of the present invention is usable for building a predictive model used to call attention for the target persons and for providing information to call attention, concerning the predetermined diseases or disorders associated with the symptom correspond to no disease but impairing the daily life, or the like.

What is claimed is:

1. A method for building a health predictive model, the method comprising:
acquiring purchase data that indicates a purchase history of each of a plurality of subjects;
acquiring symptom data that indicates an extent of a health symptom collected from each of the plurality of subjects; and
building a health predictive model that correlates therein a merchandise category to which at least one merchandise comprised in the purchase history belongs with the extent of the health symptom, by performing machine learning using the purchase data as an explanatory variable and the symptom data as an objective variable.

2. The method for building a health predictive model according to claim 1, wherein
the health symptom of each of the subjects is classified into any one of a plurality of classes according to an extent of the health symptom, and wherein
the health predictive model is built by correlating the merchandise category to which the at least one merchandise belongs and a value corresponding to number of sessions of selecting the at least one merchandise by a subject corresponding to each of the classes for each of the plurality of classes, with each other.

3. The method for building a health predictive model according to claim 1, wherein
the machine learning is machine learning by logistic regression.

4. The method for building a health predictive model according to claim 3, wherein
the logistic regression uses an L1-regularization method and/or an L2-regularization method.

5. The method for building a health predictive model according to claim 1, wherein
the merchandise category provides segmented names of items that are different from trade names of the merchandise and that are included in foods and/or daily commodities.

6. The method for building a health predictive model according to claim 1, wherein
the symptom data indicating an extent of the health symptom indicates one of:
an extent of subjective symptom at a time when the subjective symptom was collected from each of the plurality of subjects; and
an extent of the symptom that is recognized as results of measurement.

7. An information providing method comprising:
acquiring purchase data that indicates a purchase history of a target customer;
determining a merchandise category to which at least one merchandise belongs, from the purchase data;
predicting an extent of a potential health symptom of the target customer using a health predictive model correlating therein a merchandise category to which at least one merchandise comprised in the purchase history belongs and an extent of the health symptom with each other, and the merchandise category; and
providing health information that indicates the extent of the health symptom that is predicted, to the target customer.

8. The information providing method according to claim 7, wherein
the health symptom is classified into any one of a plurality of classes according to an extent of the health symptom, wherein
each of the plurality of classes is correlated in advance with a numerical value range that corresponds to the extent of the health symptom, wherein
the extent of the potential health symptom of the target customer is represented by a numerical value, and wherein
the extent of the potential health symptom of the target customer is predicted by determining a class that corresponds to the numerical value based on a numerical value range to which the numerical value belongs.

9. The information providing method according to claim 8, wherein
the plurality of classes are three or more classes.

10. The information providing method according to claim 8, wherein
a table correlating therein each of the plurality of classes and a service and/or a merchandise that adapt(s) to a health symptom of each of the classes with each other is prepared in advance, and wherein
a proposal related to the service and/or the merchandise that adapt(s) to the health symptom of the determined class, is further provided together with the health information.

11. The information providing method according to claim 10, wherein
the health information and/or the proposal are/is provided through a text in an electronic mail, a display on a displaying device, printing on a receipt, or a message presented on an app executed by an electronic device.

12. The information providing method according to claim 7, wherein
the health predictive model is built by the method for building a health predictive model according to claim 1.

13. The information providing method according to claim 7, wherein the merchandise category provides segmented names of items that are different from trade names of the merchandise and that are included in foods and/or daily commodities.

14. The information providing method according to claim 7, wherein
the symptom data indicating an extent of the health symptom indicates one of:
an extent of subjective symptom at a time when the subjective symptom was collected from each of the plurality of subjects; and
an extent of the symptom that is recognized as results of measurement.

15. The method for building a health predictive model according to claim 1, wherein
the purchase history is a purchase history of foods and/or daily commodities purchased by each of a plurality of women in a specific time period, and wherein
the health symptom is represented by any one or more of a simplified menopausal index (SMI), a Menopause Rating Scale, a Kupperman Index, Green Climacteric Scale, a simplified menopausal index (SMI), a premenstrual symptoms screening tool (PSST), Menopause Rating Scale (MRS), a Women's Health Questionnaire (WHQ), Visual Analogue Scale (VAS), Hot Flash Related Daily Interference Scale (HFRDI), Hot Flash Composite Score (HFCS), and Menopause-Specific Quality of Life (MENQOL).

16. The method for building a health predictive model according to claim 1, wherein
the health predictive model is one or more selected from a menopause predictive model and a premenstrual syndrome (PMS, PMDD) predictive model.

17. The information providing method according to claim 7, wherein
the potential health symptom of the target customer is one or more selected from a menopause symptom and a premenstrual syndrome (PMS, PMDD) symptom.

18. The method for building a health predictive model according to claim 1, wherein
the purchase history is a purchase history of foods and/or daily commodities purchased by each of the plurality of subjects in a specific time period, and wherein
the extent of the health symptom is represented by any one or more of worsening of a sleeping condition, a disordered sleeping rhythm, and an improper sleeping time period.

19. The method for building a health predictive model according to claim 1, further comprising performing the machine learning by including one or more pieces of information of a quality of sleeping of the plurality of subjects.

20. The method for building a health predictive model according to claim 1, wherein
the health predictive model is a sleeping condition predictive model.

21. The information providing method according to claim 7, wherein
the potential health symptom of the target customer is one or more selected from worsening of a sleeping condition, a disordered sleeping rhythm, and an improper sleeping time period.

22. The method for building a health predictive model according to claim 1, wherein
the purchase history is a purchase history of foods and/or daily commodities purchased by each of the plurality of subjects in a specific time period, and wherein
the extent of the health symptom is represented by any one or more of insufficient exercise, overweight, and a variation of a body weight in a specific time period.

23. The method for building a health predictive model according to claim 1, further comprising performing the machine learning by including one or more pieces of information of being a smoker or a non-smoker, and sedentary behaviors of the plurality of subjects.

24. The method for building a health predictive model according to claim 1, wherein
the health predictive model is an exercise condition predictive model related to an exercise condition.

25. The information providing method according to claim 7, wherein
the potential health symptom of the target customer is one or more selected from insufficient exercise, overweight, and a variation of a body weight in a specific time period.

26. The method for building a health predictive model according to claim 1, wherein
the purchase history is a purchase history of foods and/or daily commodities purchased by each of the plurality of subjects in a specific time period, and wherein
the extent of the health symptom is represented by any one or more of a high value of serum Na, and a high value of a BUN/creatinine ratio.

27. The method for building a health predictive model according to claim 1, further comprising performing the machine learning by including one or more pieces of information of a water intake amount, an alcohol intake amount, a body activity amount, and a color of urine.

28. The method for building a health predictive model according to claim 1, wherein
the health predictive model is a water replacement state predictive model related to a water replacement state.

29. The information providing method according to claim 7, wherein
potential health information of the target customer is one or more selected from a high value of serum Na, and a high value of a BUN/creatinine ratio.

30. The method for building a health predictive model according to claim 1, wherein
the purchase history is a purchase history of foods and/or daily commodities purchased by each of the plurality of subjects in a specific time period, and wherein
the extent of the health symptom is represented by one or more of easiness of catching a cold, an SIgA concentration, an allergic symptom, a state of an oral environment, a serious sleeping symptom, an intermediate sleeping symptom, and a mild sleeping symptom.

31. The method for building a health predictive model according to claim 1, wherein
the health predictive model is an immune state predictive model related to an immune state.

32. The information providing method according to claim 7, wherein
potential health information of the target customer is one or more selected from easiness of catching a cold, an SIgA concentration, an allergic symptom, and a state of an oral environment.

33. The method for building a health predictive model according to claim 1, wherein
the purchase history is a purchase history of foods and/or daily commodities purchased by each of the plurality of subjects in a specific time period, and wherein
the extent of the health symptom is represented by one or more of presence or absence of a subjective symptom associated with worsened nutrition, a dietary variety score (DVS), a simplified nutritional appetite questionnaire (SNAQ), a serious sleeping symptom, an intermediate sleeping symptom, and a mild sleeping symptom.

34. The method for building a health predictive model according to claim 1, wherein
the health predictive model is a nutritional state predictive model related to a nutritional state.

35. The information providing method according to claim 7, wherein
potential health information of the target customer is one or more selected from insufficient nutrition, disproportionate nutrition, and a dietary habit.
